(19) United States
(12) Patent Application Publication
(10) Pub. No.: US 2026/0204346 A1
(43) Pub. Date: Jul. 16, 2026

(54) METHOD FOR IDENTIFYING EXTRACELLULAR RNA BIOMARKERS IN BODY FLUID SAMPLES IN VIEW OF DETECTING CANCERS AND OTHER PATHOLOGIES SUCH AS ENDOMETRIOSIS

(71) Applicant: ZIWIG, Lyon (FR)

(72) Inventor: Stéphane SUISSE, Lyon (FR)

(21) Appl. No.: 19/138,091

(22) PCT Filed: Dec. 8, 2023
(86) PCT No.: PCT/EP2023/084943

§ 371 Date: Jun. 11, 2025

(30) Foreign Application Priority Data
Dec. 12, 2022 (FR) FR2213196

(51) Int. Cl. G16B 20/20; G16B 40/20; G16H 10/40; G16H 10/60; G16H 50/20

(52) U.S. Cl. CPC G16B 20/20; G16B 40/20; G16H 10/40; G16H 10/60; G16H 50/20

(57) ABSTRACT

The present disclosure relates to a method for generating a trained classifier adapted to detect a targeted pathology in a body fluid sample, the method comprising: processing a set of body fluid samples each being associated with a presence indicator of the targeted pathology, to obtain processed samples each comprising a list of exRNAs comprising values representative of occurrence numbers of the exRNAs, detected in the body fluid sample; training machine learning models using the processed samples and a presence indicator of the targeted pathology associated with the processed samples; testing the trained machine learning models; generating a list of selected exRNA as a function of the scores attributed by the trained machine learning models; and training a classifier using the processed samples and the presence indicator of the targeted pathology, the trained classifier and the list of selected exRNA being used to detect the targeted pathology.

### TECHNICAL FIELD

The present invention relates to the field of body fluid samples analysis in view of identifying extracellular RNA (RiboNucleic Acid) biomarkers that are relevant to detect a targeted pathology such as a particular cancer and endometriosis.

### BACKGROUND ART

Some extracellular RNAs (exRNA) can be detected in a variety of bodily fluids such as serum, saliva, and urine. In the following, ExRNA mainly designates messenger RNA (mRNA) fragments and various non-coding RNAs: such as micro RNA (miRNA), Piwi-interacting RNA (piRNA), small nuclear RNA (snRNA), snoRNA (Small Nucleolar RNA), siRNA (Small Interfering RNA), long non-coding RNAs (IncRNA), lincRNA (Long Intergenic Non Coding RNA), circular RNA (circRNA), tRNA (Transfer RNA), etc. These molecules are stable against RNase degradation in a variety of body fluids (serum, saliva, urine, etc.) due to their self-stabilizing structure, cell membrane-like structure and protein protection, plus the specific structure of certain RNAs. Thus, ExRNAs may be used as biomarkers for helping to detect diseases, such as cancers and other pathologies like endometriosis.

Early diagnosis of endometriosis is difficult as patients can present a variety of non-specific symptoms including dysmenorrhea, dyspareunia, chronic pelvic pain, and infertility. Despite the use of specific endometriosis screening questionnaires, the time from onset to diagnosis can take more than seven years. Moreover, a Cochrane review by Nisenblat et al. [Refs. 1, 2, 3] highlighted that imaging explorations such as transvaginal ultra-sonography and magnetic resonance imaging (MRI) have a high accuracy in diagnosing endometriosis and some deep endometriosis locations. However, they exhibit poor accuracy for detecting peritoneal endometriosis which represents the early stages of the disease. Similarly, numerous studies have evaluated the diagnostic value of blood biomarkers but with disappointing results.

Thus, the gold standard for diagnosing endometriosis remains laparoscopy. However, laparoscopy is an expensive and invasive procedure performed under general anesthesia. As the risks of laparoscopy, though rare, are serious, this forms a major barrier to primary care providers, and a cause for hesitancy among patients.

Cumulative evidence suggests that miRNA dysregulation plays a pivotal role in endometriosis, and several studies have investigated the potential diagnostic value of blood miRNAs. Human miRNAs are non-coding RNAs composed of 21-25 nucleotides which bind to their complementary mRNA, thereby regulating degradation and translation of the target gene. About 60% of genes are regulated by miRNAs. To date, more than 2600 miRNAs have been identified in the human, but only a few hundred have been evaluated in the specific setting of endometriosis. Some teams have attempted to build a blood-based miRNA signature to detect patients with endometriosis.

Using genome-wide miRNA expression profiling by small RNA sequencing from plasma available in a biobank, Vanhie et al. [Ref. 4] identified a set of 42 miRNAs with discriminative power to differentiate between patients with and without endometriosis. Expression of 41 of these miRNAs was confirmed by RT-qPCR (Quantitative Reverse Transcription Polymerase Chain Reaction) and three diagnostic models were built to discriminate between controls and all stages of endometriosis, i.e. minimal-mild endometriosis, and moderate to severe endometriosis. Only the model for minimal-mild endometriosis (miR-125b-5p, miR-28-5p and miR-29a-3p) exhibited an Area Under the Receiver Operating Characteristic (ROC) Curve (AUC) of 60%, and while its sensitivity was acceptable at 78%, the specificity was only 37%. Selecting some miRNAs altered in endometriosis from a large screen, Moustafa et al. [Ref. 5] reported increased expression of four serum miRNAs (miR-125b-5p, miR-150-5p, miR-342-3p, miR-451a) and decreased expression of two (miR-3613-5p, let-7b). The authors concluded that their 6-miRNA signature was able to differentiate patients with endometriosis from those with other gynecologic disorders with an accuracy greater than 0.915. However, overall, the studies in this field are based on small sample sizes limiting the validation of the miRNA signatures.

In addition, discrepancies in methodology (study design, collection, storage, sequencing techniques, and statistical approach) have a particularly strong influence on the results of small studies. Moreover, miRNA selection based on the highest AUC is of low accuracy since the extreme variability of the endometriosis phenotypes has a major impact on the AUC. This may explain why signatures composed of a small selection of miRNAs are of low validity, stability, and reproducibility. Thus, despite the findings of these studies, no new blood-based biomarkers are currently used in clinical practice for the diagnosis of endometriosis.

Thus, there is a need for developing a method for identifying extracellular RNA biomarkers in body fluid samples in view of detecting pathologies such as cancers and endometriosis, and for diagnosing such pathologies with a low error rate.

### DISCLOSURE OF INVENTION

The present disclosure is related to a method for generating a trained classifier adapted to detect a targeted pathology in a body fluid sample, the method comprising: processing a set of body fluid samples each being associated with a presence indicator of the targeted pathology, to obtain for each body fluid sample a first processed sample comprising a list of exRNA comprising for each exRNA of an exRNA list a value representative of an occurrence number of the exRNA, detected in the body fluid sample, each of the first processed samples being associated with the presence indicator of the targeted pathology of the corresponding body fluid sample; training each machine learning model of a list of machine learning models using a first part of the first processed samples and the presence indicator of the targeted pathology associated with the first processed samples of the first part; testing each trained machine learning model using a second part of the first processed samples comprising first processed samples that do not belong to the first part of the first processed samples, and retrieving scores attributed by the trained machine learning model to the exRNAs of the list of exRNAs when the first processed samples are used to test the trained machine learning model; generating a list of selected exRNA as a function of the scores attributed by the trained machine learning models to the exRNAs; generating from each first processed sample a corresponding second processed sample by keeping in the first processed sample exRNAs that belong to the list of selected exRNA; training a classifier using at least a part of the second processed samples and the presence indicator of the targeted pathology associated with the corresponding first processed samples, the trained classifier and the list of selected exRNA being used to detect the targeted pathology.

The selection of features performed by training and testing a number of machine learning models enables to eliminate irrelevant and noisy features and keeping the features having a maximum relevance regarding the target pathology and minimum redundancy. The combination of several machine learning models eliminates biases that may occur for a given pathology or type of exRNAs. Since the selection of features impact the training of the classifier, it further reduces the classifier complexity which makes it more explainable and more cost-effective. The selection of features further improves the classifier performances, thus avoiding overfitting and creating general models.

According to an embodiment, the trained classifier belongs to a list of classifiers, the method further comprising: training each classifier of the list of classifiers using a first part of the second processed samples each being associated with the indicator of the presence of the targeted pathology; testing each trained classifier of at least a part of the trained classifiers using a second part of the second processed samples comprising second processed samples that do not belong to the first part of the second processed samples, evaluating a performance indicator of an ability of the trained classifier to detect the targeted pathology, the trained classifier used to detect the targeted pathology being selected when the performance indicator is greater than a performance threshold.

Thus, the classifier used to detect the targeted pathology is also selected among a list of classifiers as showing the best performances for detecting the targeted pathology.

According to an embodiment, the performance indicator is AUC.

AUC has shown to be a suitable performance indicator to compare the results provided by the tested classifiers.

According to an embodiment, the list of classifiers comprises one or more of Logistic Regression classifier, Random Forest Classifier, eXtreme Gradient Boosting classifier, and Ada Boost Classifier.

These classifiers are selected as being particularly adapted to detect endometriosis from miRNAs.

According to an embodiment, the method further comprises: preprocessing the body fluid samples to obtain preprocessed samples; distributing the preprocessed samples into a number of sample groups comprising a same number of preprocessed samples; and performing a number of standardizations of the preprocessed samples to obtain the first processed samples, each standardization using a mean and a standard deviation computed from preprocessed samples of all the sample groups except a respective sample group, the number of standardizations corresponding to the number of sample groups, the number of the first processed samples being equal to a number of preprocessed sample multiplied by the number of standardizations.

When the number of body fluid samples is too small to provide statistically relevant results, the distribution of the samples into sample groups and the performance as much standardization steps as the number of sample groups enables to obtain a sufficient number of standardized samples.

According to an embodiment, the number of preprocessed samples in each sample group is set to one.

In this manner, the number of standardized samples can be very high, to obtain statistically relevant results, even when the number of body fluid samples is small, such as 50.

According to an embodiment, the method further comprises: summing the scores attributed to each exRNA by the trained machine learning models to obtain a resultant score for each exRNA, the generation of the list of selected exRNA comprising selecting the exRNAs having resultant scores greater than a score threshold.

According to an embodiment, the list of machine learning models comprises filter methods, wrapper methods and embedded methods.

The combination of these different method types to be used as machine learning models appears to be particularly efficient for selecting features.

According to an embodiment, the list of machine learning models comprises at least five of the following machine learning models: Analysis of Variance, Wilcoxon Rank Sum, Chi-Square distance metric, Pearson correlation coefficient, Recursive Feature Elimination based on Support Vector Machines, a decision tree classifier, Random Forest classifier, AdaBoost classifier, Extra tree classifier, Lasso regularized model, Ridge Regression, Stochastic Gradient Descent classifier, Gradient Boosting, and Linear Discriminative Analysis.

According to an embodiment, the exRNAs of the list of exRNA are of one of the following types: mRNA fragment, ncRNA, IncRNA, snoRNA, snRNA, siRNA, miRNA, piRNA, lincRNA, tRNA and circRNA.

Embodiments may also relate to a method for detecting a targeted pathology from a body fluid sample, comprising: analyzing a body fluid sample to obtain a processed sample comprising a list of exRNA comprising for each exRNA of the list of selected exRNA a value representative of an occurrence number of the exRNA, detected in the body fluid sample; filtering the processed sample to remove exRNAs that do not belong to a list of selected exRNAs; and executing a trained classifier on the filtered processed sample, the execution of the trained classifier providing an indicator of the presence or absence of the targeted pathology, wherein the list of selected exRNAs and the trained classifier are generated by the method according to one of claims 1 to 10.

The detection of the targeted pathology is performed by a classifier that is trained using a list of exRNAs selected as being relevant for the targeted pathology. In addition, the trained classifier can be also selected for its efficiency with regard to its effeciency for detecting the targeted pathology.

Embodiments may also relate to a system comprising: an analyzing unit for producing from body fluid samples preprocessed samples associating to each exRNA of a list of exRNAs values representative of an occurrence number of the exRNA detected in the body fluid sample; and a processor configured to generate from the preprocessed samples a trained classifier adapted to detect a targeted pathology in a body fluid sample, the system being configured to implement the method as disclosed above.

Embodiments may also relate to a computer program product comprising program code for performing, when executed by one or more processors, the method as disclosed above.

### BRIEF DESCRIPTION OF DRAWINGS

The foregoing and other purposes, features, aspects and advantages of the invention will become apparent from the following detailed description of embodiments, given by way of illustration and not limitation, with reference to the accompanying drawings, in which the same reference refer to similar elements or to elements having similar functions. In the accompanying drawings:

FIG. 1 is a flow chart diagram showing steps of a method for identifying extracellular RNA biomarkers in body fluid samples and generating a trained classifier, according to an embodiment;

FIG. 2 is a schematic representation of a sample processed by the identification method, according to an embodiment;

FIG. 3, FIG. 4 and FIG. 5 are flow chart diagram detailing steps of the identification method, according to embodiments;

FIG. 6 is a flow chart diagram showing steps of a method for detecting a particular pathology, using a classifier trained by the identification method, according to an embodiment.

FIG. 7 is a schematic diagram of a computer system for implementing the methods disclosed herein, according to an example.

### BEST MODE FOR CARRYING OUT THE INVENTION

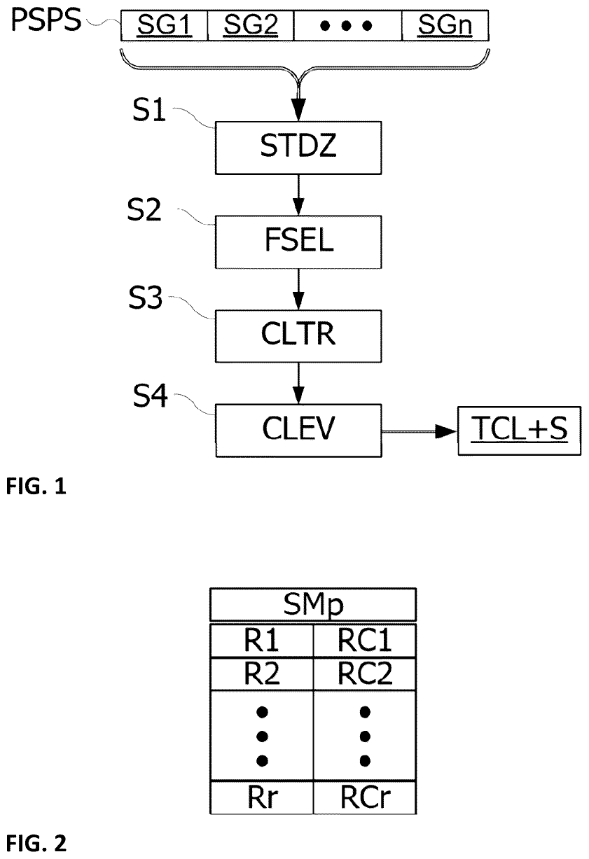

FIG. 1 shows general steps S1-S4 of a method for identifying extracellular RNA (exRNA) biomarkers in body fluid samples for a particular targeted pathology, according to an embodiment. The method receives preprocessed samples PSPS collected from patients having symptoms of the target pathology. All the patients were submitted to further analyses to confirm the presence or absence of the targeted pathology.

In the case of endometriosis, the selected patients suffer from pelvic pain. Some of the patients underwent a laparoscopic procedure (either therapeutic or diagnostic laparoscopy) and/or MRI imaging. For all the patients undergoing a laparoscopy, a systematic video was performed and analyzed by two operators (CT, YD) blind of the symptoms and imaging findings to confirm the presence or absence of endometriosis. For those patients, the diagnosis was confirmed by histology. For the patients without laparoscopic evaluation in the endometriosis group where the presence of endometriosis was confirmed, all had an MRI with features of deep endometriosis with colorectal involvement and/or endometrioma confirmed by two expert radiologists. Mi-RNA analysis was performed blinded to the surgical and imaging findings.

Preprocessing of body fluid samples may comprise FASTQ sequencing generating FASTQ files. The FASTQ files are trimmed to remove adapter sequences using Cutadapt software and are aligned using Bowtie software to the following transcriptome databases: the human reference genome available from NCBI (https://www.ncbi.nlm.nih.gov/genome/guide/human/), and miRBase21 (miRNAs) using the MirDeep2 package. A raw sequencing data quality is assessed using FastQC software.

An expression level quantification of miRNA can be determined by miRDeep2 software. Then, differential expression tests of the samples using DESeq2 software are conducted only for miRNA with read counts exceeding or equal to 1. Software DESeq2 integrates processes with several features to facilitate a more quantitative analysis of comparative RNA-seq data using shrinkage estimators for dispersion and fold change. Software DESeq2 provides a matrix which is filtered for expressed miRNAs. MiRNAs were considered as differentially expressed if the absolute value of log2-fold change was >1.5 (up) and <0.5 (down) and the P value adjusted for multiple testing was <0.05.

According to the miRBase21 library, each sample may comprise 2561 different miRNA called “features” that require two adjustments to avoid experimental biases with library preparation followed by PCR amplification (DESeq2 normalization), and normalization to avoid feature importance biases in machine learning models during the training phase. DESeq2 normalization performs an internal normalization where a geometric mean is computed for each miRNA across all samples. The occurrences of each miRNA are counted in each sample and each miRNA count is divided by the corresponding geometric mean computed for the sample. A size factor is defined for each sample as the median of these ratios. This normalization corrects for library size and RNA composition biases which may arise for example when only a small number of miRNAs are very highly expressed in one experiment condition but not in others.

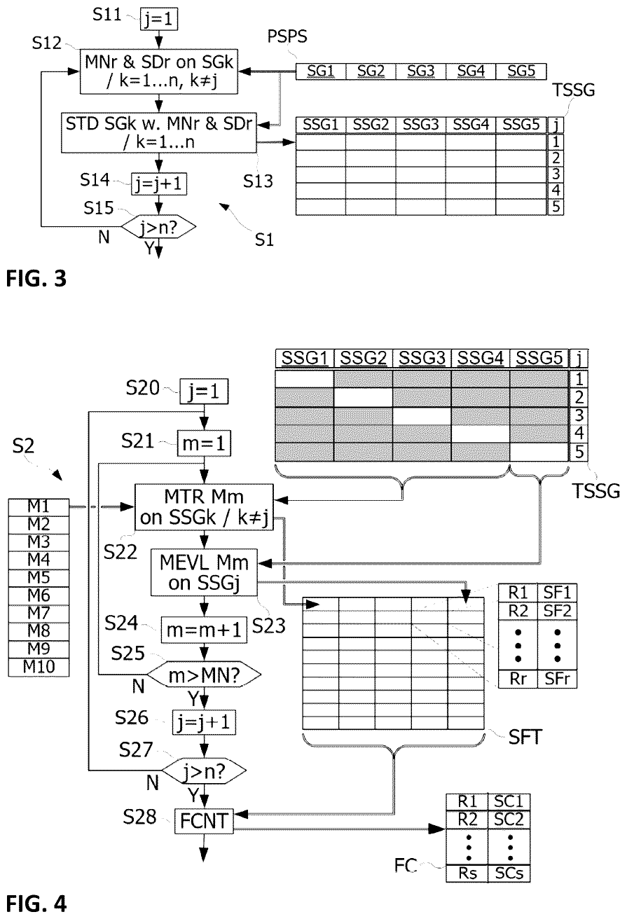

FIG. 2 shows a preprocessed sample SMp, according to an embodiment. The sample SMp is a list of a number of r miRNA labels R1, R2, . . . , Rr, each miRNA being associated with a number RC1, RC2, . . . , RCr representative of an occurrence number of the miRNA detected in the corresponding original body fluid sample. The miRNAs R1, R2, . . . , Rr are for example the 2561 miRNA listed in the miRBase21 library. For example, the miRNA labels R1, R2, . . . Rr may have the form “hsa-let-7a-2-3p”, . . . , “hsa-let-7i-5p”, “hsa-miR-1-3p”, . . . , “hsa-miR-99b-5p”.

In step S1, the preprocessed samples PSPS are standardized by applying a feature standardization technique. In step S2, features are selected using machine learning models that are trained on parts of the standardized samples and evaluated on other parts of the standardized samples. In step S3, classifier models are trained on parts of the standardized samples using the features selected at step S2. In step S4, the trained classifiers are evaluated on another part of the standardized samples using the features selected at step S2. Thus step S4 provides a trained classifier TCL and a set of features (miRNAs) forming a signature S and that are selected to be analyzed using the selected trained classifier to provide a diagnosis of the target pathology.

According to an embodiment, the preprocessed samples PSPS are distributed into sample groups SG1, SG2, . . . SGn of one or more preprocessed samples, each sample group SGi (i=1, 2, . . . n) comprising a same number of samples.

In step S1, the feature standardization technique makes the ratios resulting from sample normalization have a zero-mean and a standard deviation of 1. Such a standardization enables to compare measurements that may have different units and avoids feature importance biases. The standardization can be performed using Z-Score (or Standard Score) algorithm. According to an embodiment, this algorithm is applied a number of iterations equal to the number n of sample groups SGi. At each iteration, one respective sample group SGi is selected, a mean and a standard deviation is computed from all the samples belonging to the n-1 non-selected sample groups, and all the samples are standardized using the computed mean and standard deviation. Thus, step S1 produces n sample sets of n sample groups of standardized samples, each sample set being associated with one respective sample group SGi selected at one corresponding iteration. In each sample set of standardized samples, each of the selected features S in each sample is associated with a respective value having a mean equal to zero and a standard deviation equal to one with regard to a respective sample set including all the sample groups SG1-SGn except the sample group selected at the corresponding iteration.

The body fluids samples comprise in substantively identical proportions positive, negative and complex cases regarding the targeted pathology. According to an example, 200 samples are distributed into 5 sample groups SG1-SG5 of 40 samples. Thus Z-Score (or Standard Score) is applied 5 times to standardize all the samples at each iteration using a mean and standard deviation computed on 160 samples (n-1 sample groups). According to another example, the sample groups comprise a respective single sample. Therefore, when 200 samples are available, there are 200 sample groups and the Z-score standardization is applied 200 times to produce 200×200 standardized samples.

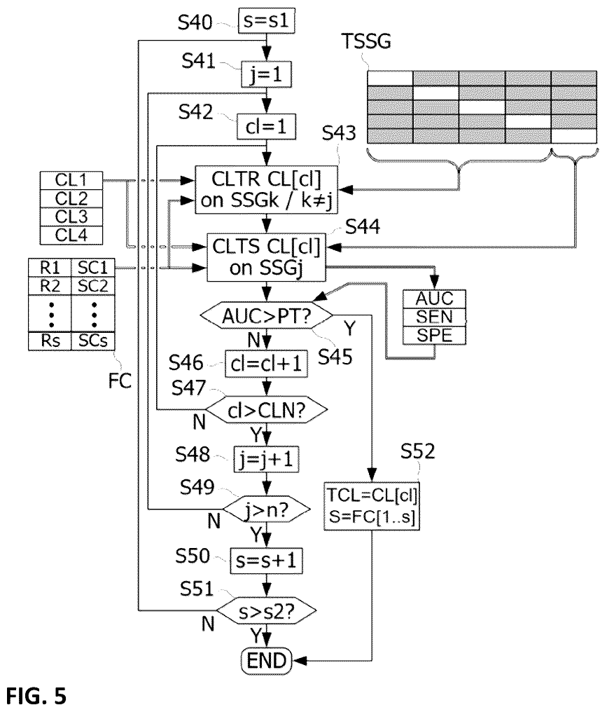

FIG. 3 shows steps S11 to S15 performed during execution of step S1, according to an example. In step S11, an index j is initialized to one. In step S12, a mean MNr and a standard deviation SDr is computed for each miRNA r, on all values of the miRNA r of the samples of all the sample groups SG1-SGn (n=5 in the example of FIG. 3), except the sample group SGj selected by index j. In step S13, the miRNA values of each miRNA r of the samples of all the sample groups SG1-SGn are standardized using the computed mean MNr and standard deviation SDr computed for the miRNA r, according to the Z-score algorithm. Step S13 provides standardized samples distributed into groups SSGi, j. In steps S14 and S15, index j is incremented by one and compared with the number n of sample groups SGi. In step S15, if index j is not greater than number n, a new iteration including steps S12 to S15 is executed, otherwise the execution of step S1 ends.

Thus, step S1 provides a table TSSG comprising n x n standardized sample groups SSGi, j obtained with different sets of one mean MNr and standard deviation SDr per miRNA r.

FIG. 4 shows steps S20 to S28 performed during execution of step S2, according to an example. Steps S20 to S25 are executed successively for a first iteration. In steps S20 and S21, indexes j and m are initialized to one. In step S22, n-1 standardized sample group SSG1-SSGn in table TSSG, except the sample group SSGj selected by index j, are used to train one machine learning model Mm selected by index m in a list of machine learning models M1-M10. To train the machine learning models, each of the samples in the sample groups SSG1-SSGN is associated with an indicator specifying the confirmed presence or absence of the targeted pathology. The result provided at the end of training of the model Mm comprises lists of the r miRNAs R1-Rr respectively associated with scores SF1-SFr. These lists are stored in a table SFT at indexes k, m with k=1 to n and k different from index j. In step S23, the trained model Mm is evaluated using the sample set SSGj selected by index j or all sample sets SSG1-SSGn. The result provided at the end of evaluation of the model Mm comprises one list of the r miRNAs respectively associated with scores SF1-SFr. This list is stored in table SFT at indexes j, m. All the lists stored in table SFT are ordered by decreasing scores and the scores below a preset threshold value or the scores corresponding to miRNA having a rank in the ordered lists smaller than a preset threshold value are set to zero. For example, only the scores of the 100 first ranked miRNA are kept in each list stored in table SFT.

In steps S24, S25, index m is incremented by one and compared with the number MN of machine learning models M1-M10 to be trained and evaluated. In step S25, if index m is not greater than number MN of machine learning models M1-M10, a new iteration is executed from step S22, otherwise steps S26 and S27 are executed. In steps S26, S27, index j is incremented by one and compared with the number n of sample groups SSGi in table TSSG. In step S27, if index j is not greater than number n, a new iteration is executed from step S21, otherwise step S28 is executed. In step S28, all scores stored in table SFT for each remaining miRNA are summed to obtain a table FC associating each miRNA R1-Rs selected at the end of step S23 with a respective resulting score SC1-SCs. Table FC may be ordered by decreasing scores. The resulting scores SC1-SCs can be further divided by MN×n. Then the execution of step S2 ends.

The number of machine learning models M1-M10 to be trained and evaluated at steps S22, S23 can set between 8 and 12. The machine learning models M1-M10 can comprise:
filter methods such as ANOVA (Analysis of Variance Ref [6]), Wilcoxon Rank Sum (Ref [7]), Chi-Square distance metric and Pearson correlation coefficient (Ref [8]),
wrapper methods such as RFE (Recursive Feature Elimination) based on SVC (Support Vector Machines) (Ref [9]), or on a decision tree classifier (Ref [10]),
embedded methods which comprise classifiers such as Random Forest (Ref [11]), AdaBoost (Ref [12]), Decision tree classifier (Ref [17]) and Extra tree classifier (Ref [18]), and regularized models such as Lasso (Ref) [13]), Ridge Regression (Ref [14]), SGD (Stochastic Gradient Descent) classifier (Ref [15]), Gradient Boosting (Ref [16]), and
hybrid methods such as LDA (Linear Discriminative Analysis) (Ref [19]).

According to Chi-Square distance metric method, the chi-square metric is calculated between a target (diagnosis) and a numerical variable (the miRNA normalized count). If the target variable is independent of the feature, then it gets a low score, otherwise, the feature (or miRNA) is important. A higher value of chi-square means that the feature is more relevant concerning the class.

According to the Pearson correlation coefficient method, a correlation coefficient measures the degree of the statistical linear relationship between two numerical variables. The value of the correlation coefficient could be any value between −1 and 1, a perfect negative linear relationship, and a perfect positive linear relationship, respectively. A coefficient close to 0 means that the two variables are not linearly correlated.

Wrapper methods use a machine learning algorithm as a black box estimator to find the best subsets of features (or miRNAs). Thus, they are dependent on the classifier. Two recursive feature elimination (RFE) methods can be used with two different classifiers (Logistic Regression and Decision Tree Classifier). The features are selected by recursively considering smaller and smaller sets of features. First, the estimator is trained on the initial set of features and the importance of each feature is computed. Then, the least important features are pruned from current set of features. This procedure is recursively repeated on the pruned set until the desired number of features to select is eventually reached.

Embedded methods achieve both model fitting and feature selection at the same time. They do not perform iteration. They pick a feature subset with the best classification performance. The classifiers used in embedded methods belong to ensemble methods that use decision tree estimator algorithms. Feature importance scores are based on the reduction in the criterion used to select split points, like “gini” or “entropy”.

Embedded methods using regularized linear models are a powerful set of tools for feature interpretation and selection. Among them, Lasso gives a penalty for having many features by making some unimportant features exactly zero. It uses the Logistic Regression Classifier with L1 penalization. Ridge adds the L2 norm penalty to a loss function. In this manner, useful features tend to have non-zero coefficients. Stochastic Gradient Descent Classifier uses a regularized (L2) linear SVM (support vector machine) model with stochastic gradient descent (SGD) learning. Thus the gradient of the loss is estimated for each sample at a time and the model is updated along the way with a decreasing strength schedule or learning rate.

Linear Discriminative Analysis (LDA) is a dimension reduction technique, to select features which are more useful for predicting the target variable. It is a learning-based model and compared with wrapper-based feature selection, LDA does not require to recursively call the learning model.

FIG. 5 shows steps S40 to S52 performed during execution of steps S3 and S4. Steps S40-S46 are executed successively. In step S40, an index s is initialized to a value s1. Value s1 represents a minimum number of miRNAs to be taken into account in the ordered list FC from the beginning thereof in the following computations. In steps S41, S42, indexes j and cl are successively initialized to one. In step S43, n-1 standardized sample sets SSG1-SSGn in table TSSG, except the sample set SSGj selected by index j, are used to train one classifier CL[cl] selected by index cl in a list of classifiers CL1-CL4. Training the classifier CL[cl] takes into account the s first miRNAs as defined in the ordered list FC when using the standardized sample sets SSG1-SSGn.

In step S44, the trained classifier CL[cl] is evaluated using the sample set SSGj selected by index j and more specifically the miRNA values in the sample set SSGj as defined by the s first miRNAs in the ordered list FC. The evaluation of the trained classifier CL[cl] can provide performance indicators such as AUC (Area Under the Curve), sensitivity SEN and specificity SPE. To provide such performance indicators AUC, SEN, SPE, a confusion matrix is generated on the basis of predictions produced by the trained classifier CL[cl] applied to each sample in table TSSG. Such a confusion matrix comprises four counter values of the samples detected as true and false positive cases and true and false negative cases.

In step S45, one or more of the performance indicators AUC, SEN, SPE computed in step S44 are compared with threshold values. If the performance indicators show that the classifier CL[cl] reaches an expected performance, the classifier CL[cl] (=TCL in FIG. 1) is selected together with the list part FC[1 . . . s] (=S in FIG. 1) comprising the s first miRNAs of the ordered list FC, at step S52. In the example of FIG. 5, the performance indicator AUC is compared at step S45 with a threshold value PT that can be set to 0.95.

In steps S46, S47, index cl is incremented by one and compared with the number CLN of classifiers CL1-CL4 to be trained and evaluated. In step S47, if index cl is not greater than number CLN of classifiers CL1-CL4, a new iteration is executed from step S43, otherwise steps S48 and S49 are executed. In steps S48, S49, index j is incremented by one and compared with the number n of sample groups SSGi in table TSSG. In step S49, if index j is not greater than number n, a new iteration is executed from step S42, otherwise steps S50, S51 are executed. In steps S50, S51, index s is incremented by one and compared with a maximum number s2 of first miRNAs to be taken into account in table FC during the execution of steps S43 and S44. In step S51, if index s is not greater than the maximum number s2, a new iteration is executed from step S41, otherwise it is considered that none of the classifiers CL1-CL4 applied to not too many miRNAs (≤s2) provides an exploitable result for detecting a targeted pathology in a body fluid sample. In an example, s1 is set to 10 and s2 is set to 120.

The list of classifiers CL1-CL4 may comprise the following classifiers: Logistic Regression classifier, Random Forest Classifier, eXtreme Gradient Boosting classifier, and Ada Boost Classifier.

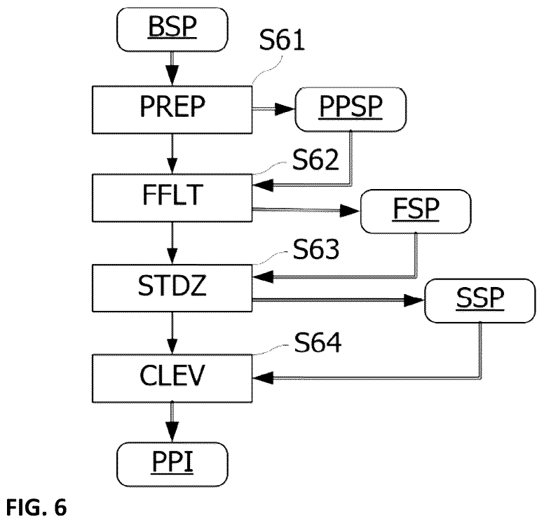

FIG. 6 shows steps S61-S64 of a method for detecting the targeted pathology from a body fluid sample, according to an embodiment. In step S61, a body fluid sample BSP is preprocessed. Such a preprocessing can be the same as the one used for obtaining the preprocessed samples PSPS. In step S62, the preprocessed sample PPSP in the form as shown in FIG. 2, is filtered to remove the miRNAs that are not listed in the list FC[1 . . . s] provided at step S52. The filtered sample FSP is standardized in step S63. This standardization step can be the same as in step S1 (FIG. 3). In step S64, the standardized sample SSP is processed by the classifier CL[cl] trained and evaluated in steps S43 and S44, and identified in step S45. The classifier CL[cl] applied to the standardized sample SSP produces an indicator PPI specifying a probability that the targeted pathology is detected in the body fluid sample BSP.

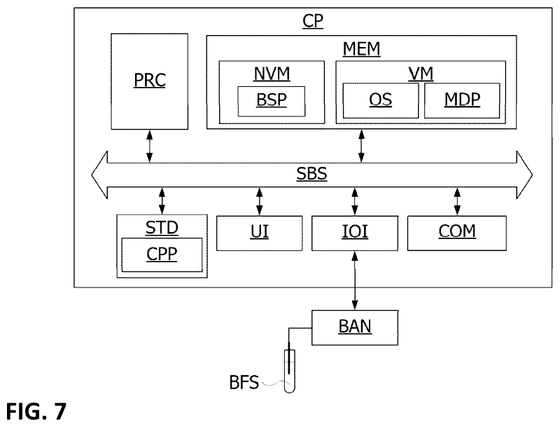

FIG. 7 shows a computer system CP for implementing examples disclosed herein. The computer system CP is adapted to execute instructions from a computer-readable medium to perform the methods described herein. The computer system CP may be connected (e.g., networked) to other machines in a LAN, an intranet, an extranet, or the Internet. While only a single device is illustrated, the computer system CP may include any collection of devices that individually or jointly execute a set (or multiple sets) of instructions to perform any one or more of the methods disclosed herein.

The computer system CP may include a processor PRC, a memory MEM, and a system bus SBS. The system bus SBS provides an interface for system components including, but not limited to, the memory MEM and the processor PRC. The processor PRC may include any number of hardware components for conducting data or signal processing or for executing computer code stored in memory MEM. The processor PRC may, for example, include a general-purpose processor, an application specific processor, a Digital Signal Processor (DSP), an Application Specific Integrated Circuit (ASIC), a Field Programmable Gate Array (FPGA), a circuit containing processing components, a group of distributed processing components, a group of distributed computers configured for processing, or other programmable logic device, discrete gate or transistor logic, discrete hardware components, or any combination thereof designed to perform the functions described herein. The processor device may further include computer executable code that controls its operation.

The system bus SBS may be any of several types of bus structures that may further interconnect to a memory bus (with or without a memory controller), a peripheral bus, and/or a local bus using any of a variety of bus architectures. The memory MEM may be one or more devices for storing data and/or computer code for implementing or facilitating methods described herein. The memory MEM may include database components, object code components, script components, or other types of information structure for supporting the various activities herein. Any distributed or local memory device may be utilized with the systems and methods of this description. The memory MEM may be communicably connected to the processor device PRC (e.g., via a circuit or any other wired, wireless, or network connection) and may include computer code for executing one or more steps described herein. The memory MEM may include non-volatile memory NVM (e.g., read-only memory (ROM), erasable programmable read-only memory (EPROM), electrically erasable programmable read-only memory (EEPROM), etc.), and volatile memory VM (e.g., random-access memory (RAM)), or any other medium which can be used to carry or store desired program code in the form of machine-executable instructions or data structures and which can be accessed by a computer or other machine with a processor device. A basic input/output system (BIOS) BSP may be stored in the non-volatile memory NVM and can include the basic routines that help to transfer information between elements within the computer system CP.

The computer system CP may further include or be coupled to a non-transitory computer-readable storage medium such as the storage device STD, which may comprise, for example, an internal or external hard disk drive (HDD) for storage, flash memory, or the like. The storage device STD may provide non-volatile storage of data, data structures, computer-executable instructions, and the like.

A number of modules can be implemented as software and/or hard-coded in circuitry to implement the functionality described herein in whole or in part. The modules may be stored in the storage device STD and/or in the volatile memory VM, which may include an operating system OS and/or one or more program modules MDP. All or a portion of the examples disclosed herein may be implemented as a computer program product CPP stored on a transitory or non-transitory computer-usable or computer-readable storage medium (e.g., single medium or multiple media), such as the storage device STD, which includes complex programming instructions (e.g., complex computer-readable program code) to cause the processor PRC to carry out the steps described herein. Thus, the computer-readable program code can comprise software instructions for implementing the functionality of the examples described herein when executed by the processor PRC. The processor PRC may serve as a controller or control system for the computer system CP that is to implement the functionality described herein.

The computer system CP also may include a user interface UI which may be configured to receive input and selections to be communicated to the computer system CP when executing instructions, from input devices (e.g. a keyboard, mouse, touch-sensitive surface, etc). Such input devices may be connected to the processor PRC through the user interface UI coupled to the system bus SBS or other interfaces such as a parallel port, a serial port, a Universal Serial Bus (USB) port, an IR interface, and the like. The user interface UI may be connected to output devices, such as to a display. The computer system CP may also include a communications interface COM suitable for communicating with a network as appropriate or desired.

The computer system CP also may include an interface circuit IOI connected to the processor PRC through the system bus SBS. The interface circuit IOI may be connected to biochemical analysis devices, for analyzing body fluid samples BFS and identifying and counting extracellular RNA (exRNA) present in the body fluid samples.

The operational steps described herein provide examples. These steps may be performed by hardware components, may be embodied in machine-executable instructions to cause a processor to perform the steps, or may be performed by a combination of hardware and software. Although a specific order of method steps may be shown or described, the order of the steps may differ. In addition, two or more steps may be performed concurrently or with partial concurrence.

The above description of various embodiments is provided for purpose of description to one of ordinary skills in the related art. It is not intended to be exhaustive or to limit the scope of the present disclosure solely to the disclosed embodiments. Numerous alternatives or variations to the present disclosure will be apparent to those of ordinary skills in the related art. Accordingly, while some alternatives or embodiments have been presented specifically, other embodiments will be apparent or easily developed by those of ordinary skills in the related art. Limitations in the appended claims should be interpreted broadly based on the language used in the claims and such limitations should not be restricted to the specific examples described above.

In this respect, it is apparent to those of ordinary skills in the related art that the present disclosure can be applied to any pathology or disease. However, according to the above specification of step S45, it may happen that none of the trained and tested classifiers CL1-CL4 provides an exploitable result. The present disclosure describes a method exploiting miRNAs. However, this method can use any extra cellular RNAs exRNA as listed above.

The treatments applied to the body fluid samples are not limited to the above examples, provided that the preprocessed samples used to train the machine learning models are comparable and do not induce statistical biases. The distribution of the processed samples into sample groups SGi and the generation of table TSSG may be performed when the number of available body fluid samples is statistically insufficient to provide suitable results. In this respect, when the number of available body fluid samples is sufficient, the standardization performed at step S13 can be performed once on each preprocessed sample PSPS and stored in table TSSG. In this case, a single iteration with index j equal to one is performed in the steps of FIGS. 4 and 5.

Other computations can be implemented to select exRNAs from the scores attributed by the trained machine learning models. For this purpose, many feature selection methods are suitable. For instance, using Mutual Information (MI) or Information Gain (IG), the algorithm will compute the amount of information that one random variable shares with another random variable. Scores are used for feature selection. Other examples of suitable feature selection methods are disclosed in refs. [22] and [23], In ref [22]. a feature score is calculated for each feature which can then be used to rank and select top scoring features for feature selection.

### INDUSTRIAL APPLICABILITY

The invention is susceptible of industrial application in detection of targeted pathologies.

### CITED REFERENCES

1. Nisenblat, V., Bossuyt, P. M., Farquhar, C., Johnson, N. & Hull, M. L., “Imaging modalities for the non-invasive diagnosis of endometriosis”, Cochrane Database Syst. Rev., https @://doi.org/10.1002/14651858.CD009591.pub2 (2016)
2. Nisenblat, V. et al., “Combination of the non-invasive tests for the diagnosis of endometriosis”, Cochrane Database Syst. Rev., https://doi.org/10.1002/14651858.CD012281 (2016)
3. Nisenblat, V. et al., “Blood biomarkers for the non-invasive diagnosis of endometriosis”, Cochrane Database Syst. Rev., https://doi.org/10.1002/14651858.CD012179 (2016)
4. Vanhie, A. O. D. et al., “Plasma miRNAs as biomarkers for endometriosis”, Hum. Reprod. Oxf. Engl,. 34(9), 1650-1660, https://doi.org/10.1093/humrep/dez116 (2019).
5. Moustafa, S. et al., “Accurate diagnosis of endometriosis using serum microRNAs”, Am. J. Obstet. Gynecol. 223(4), 557.e 1-557.e11., https://doi org/10.1016/j.ajog.2020.02.050 (2020).
6. ANOVA-https://en.wikipedia.org/wiki/Analysis_of_variance
7. Chris Wild, “The Wilcoxon Rank-Sum Test”, University of Auckland, https://www.stat.auckland.ac.nz/~wild/ChanceEnc/Ch10.wilcoxon.pdf
8. Pearson correlation coefficient https://en.wikipedia.org/wiki/Pearson_correlation_coefficient
9. H. Sanz, C. Valim, E. Vegas, J. M. Oller, F. Reverter, “SVM-RFE: selection and visualization of the most relevant features through non-linear kernels”, BMC Bioinformatics (2018) 19: 432
10. RFE on a decision tree classifier: https://scikit-learn.org/stable/modules/generated/sklearn.feature_selection.RFE.html and https://ieeexplore.ieee.org/document/8614039
11. I. Logunova, “Random Forest Classifier: Basic Principles and Applications”, https://serokell.io/blog/random-forest-classification
12. AdaBoost, https://en.wikipedia.org/wiki/AdaBoost
13. Lasso, https://en.wikipedia.org/wiki/Lasso_(statistics)
14. Ridge Regression, https://en.wikipedia.org/wiki/Ridge_regression
15. SGD Classifier https://scikit-learn.org/stable/modules/generated/sklearn.linear_model.SGDClassifier.html?highlight=sgdclassifier#sklearn.linear_model. SGDClassifier
16. Mason L., Baxter J., Bartlett P. L., Frean M. (1999). “Boosting Algorithms as Gradient Descent”, In S. A. Solla and T. K. Leen and K. Müller (ed.). Advances in Neural Information Processing Systems 12. MIT Press. pp. 512-518.
17. Rokach L., Maimon O. (2014). “Data mining with decision trees: theory and applications”, 2nd Edition. World Scientific Pub Co Inc. doi: 10.1142/9097
18. Extra tree classifier: https://scikit-learn.org/stable/modules/generated/sklearn.ensemble.ExtraTreesClassifier.html
19. Linear Discriminative Analysis: https://scikit-learn.org/stable/modules/generated/sklearn.discriminant_analysis.LinearDiscriminantAnalysis.html
20. Tolles J., Meurer W. J. (2016). “Logistic Regression Relating Patient Characteristics to Outcomes”. JAMA. 316 (5): 533-4. doi: 10.1001/jama.2016.7653
21. Extra tree classifier: https://scikit-learn.org/stable/modules/generated/sklearn.ensemble.ExtraTreesClassifier.html
22. T. Chen, T. He, “XGBoost: extreme Gradient Boosting”, Package Version: 0.6-4, Jan. 4, 2017, https://cran.microsoft.com/snapshot/2017-12-11/web/packages/xgboost/vignettes/xgboost.pdf
23. Relief (feature selection),
   https://en.wikipedia.org/wiki/Relief_(feature_selection)
24. K. R Kavitha; Avani Prakasan; P. J Dhrishya, “Score-Based Feature Selection of Gene expression Data for Cancer Classification”, Proceedings of the Fourth International Conference on Computing Methodologies and Communication (ICCMC 2020)

1. A method for generating a trained classifier (CL[cl]) adapted to detect a targeted pathology in a body fluid sample (BSP), the method comprising:
processing a set of body fluid samples each being associated with a presence indicator of the targeted pathology, to obtain for each body fluid sample a first processed sample (SSG1-SSGn) comprising a list of exRNA comprising for each exRNA of an exRNA list (R1-Rr) a value (RC1-RCr) representative of an occurrence number of the exRNA, detected in the body fluid sample, each of the first processed samples being associated with the presence indicator of the targeted pathology of the corresponding body fluid sample;
training each machine learning model of a list of machine learning models (M1-M10) using a first part (SSGk, k/j) of the first processed samples and the presence indicator of the targeted pathology associated with the first processed samples of the first part;
testing each trained machine learning model using a second part (SSGj) of the first processed samples comprising first processed samples that do not belong to the first part of the first processed samples, and retrieving scores (SF1-SFr) attributed by the trained machine learning model to the exRNAs of the list of exRNAs when the first processed samples are used to test the trained machine learning model;
generating a list (FC) of selected exRNA as a function of the scores attributed by the trained machine learning models to the exRNAs;
generating from each first processed sample a corresponding second processed sample (SFT) by keeping in the first processed sample exRNAs that belong to the list of selected exRNA;
training a classifier (CL[cl]) using at least a part (SSGk, kj) of the second processed samples and the presence indicator of the targeted pathology associated with the corresponding first processed samples, the trained classifier and the list of selected exRNA being used to detect the targeted pathology.

2. The method according to claim 1, wherein the trained classifier (CL[cl]) belongs to a list of classifiers (CL), the method further comprising:
training each classifier of the list of classifiers using a first part (SSGk, kj) of the second processed samples (SFT) each being associated with the indicator of the presence of the targeted pathology;
testing each trained classifier of at least a part of the trained classifiers using a second part (SSGj) of the second processed samples comprising second processed samples that do not belong to the first part of the second processed samples, evaluating a performance indicator (AUC) of an ability of the trained classifier to detect the targeted pathology, the trained classifier used to detect the targeted pathology being selected when the performance indicator is greater than a performance threshold (PT).

3. The method according to claim 2, wherein the performance indicator is the Area Under the Curve (AUC).

4. The method according to claim wherein the list of classifiers (CL) comprises one or more of Logistic Regression classifier, Random Forest Classifier, eXtreme Gradient Boosting classifier, and Ada Boost Classifier.

5. The method according to claim 1, further comprising:
preprocessing the body fluid samples to obtain preprocessed samples;
distributing the preprocessed samples into a number of sample groups (SG1-SGn) comprising a same number of preprocessed samples; and
performing a number of standardizations of the preprocessed samples to obtain the first processed samples (TTSG), each standardization using a mean and a standard deviation computed from preprocessed samples of all the sample groups except a respective sample group, the number of standardizations corresponding to the number of sample groups, the number of the first processed samples being equal to a number of preprocessed sample multiplied by the number of standardizations.

6. The method according to claim 5, wherein the number of preprocessed samples in each sample group is set to one.

7. The method according to claim 1, further comprising summing the scores (SF1-SFr) attributed to each exRNA (R1-Rr) by the trained machine learning models (M1-M10) to obtain a resultant score (SC1-SCr) for each exRNA, the generation of the list of selected exRNA (FC) comprising selecting the exRNAs having resultant scores greater than a score threshold.

8. The method according to claim 1, wherein the list of machine learning models (M1-M10) comprises filter methods, wrapper methods and embedded methods.

9. The method according to claim 1, wherein the list of machine learning models (M1-M10) comprises at least five of the following machine learning models:
Analysis of Variance, Wilcoxon Rank Sum,
Chi-Square distance metric,
Pearson correlation coefficient,
Recursive Feature Elimination based on Support Vector Machines,
a decision tree classifier,
Random Forest classifier,
AdaBoost classifier,
Extra tree classifier,
Lasso regularized model,
Ridge Regression,
Stochastic Gradient Descent classifier,
Gradient Boosting, and
Linear Discriminative Analysis.

10. The method according to claim 1, wherein the exRNAs of the list of exRNA (R1-Rr) are of one of the following types: mRNA fragment, ncRNA, IncRNA, snoRNA, snRNA, siRNA, miRNA, piRNA, lincRNA, tRNA and circRNA.

11. A method for detecting a targeted pathology from a body fluid sample (BSP), comprising:
analyzing a body fluid sample to obtain a processed sample (PRSP) comprising a list of exRNA comprising for each exRNA of the list of selected exRNA a value representative of an occurrence number of the exRNA, detected in the body fluid sample;
filtering the processed sample to remove exRNAs that do not belong to a list of selected exRNAs (FC[1 . . . s]); and
executing a trained classifier (CL[cl]) on the filtered processed sample (FSP), the execution of the trained classifier providing an indicator (PPI) of the presence or absence of the targeted pathology, wherein the list of selected exRNAs and the trained classifier are generated by the method according to one claim 1.

12. A system comprising:
an analyzing unit (BAN) for producing from body fluid samples (BFS) preprocessed samples (SSG1-SSGn) associating to each exRNA of a list of exRNAs values (RC1-RCr) representative of an occurrence number of the exRNA detected in the body fluid sample; and
a processor (CP) configured to generate from the preprocessed samples a trained classifier (CL[cl]) adapted to detect a targeted pathology in a body fluid sample, the system being configured to implement the method according to claim 1.

13. A computer program product comprising program code for performing, when executed by one or more processors, the method according to claim 1.
